(19) United States
(12) Patent Application Publication
(10) Pub. No.: US 2025/0000489 A1
(43) Pub. Date: Jan. 2, 2025

(54) ULTRASONIC PROBE ROTATION DEVICE

(71) Applicant: ULTRASOUND BIOTECHNOLOGY (SHANGHAI) CO., LTD., Shanghai (CN)

(72) Inventors: Yingnan ZHANG, Shanghai (CN); Minmin ZHOU, Shanghai (CN); Houliang SONG, Shanghai (CN); Pengcheng ZHU, Shanghai (CN)

(21) Appl. No.: 18/682,108

(22) PCT Filed: Feb. 17, 2023
(86) PCT No.: PCT/CN2023/076713

§ 371 Date: Feb. 7, 2024

(30) Foreign Application Priority Data
Feb. 22, 2022 (CN) 202220360034.1

(51) Int. Cl. A61B 8/00

(52) U.S. Cl. CPC A61B 8/4461

(57) ABSTRACT

The present disclosure provides an ultrasonic probe rotation device including a first connector and a second connector. An end of a shaft sleeve of the first connector is provided with several limiting grooves, and top ends of adjacent ones of the limiting grooves are coupled by a guide surface. During the rotation of a rotating inner core of the second connector, due to the guide surface is inclined to an axis of the shaft sleeve, an end of the rotating inner core is configured to abut against the guide surface and then moves toward the limiting groove by the guide surface and is locked into the limiting groove to facilitate quick installation and mating and to avoid occurrence of rotational separation.

The present application claims priority to a Chinese patent application No. CN202220360034.1 filed to China National Intellectual Property Administration on Feb. 22, 2022 and entitled “ULTRASONIC PROBE ROTATION DEVICE”.

### FIELD OF TECHNOLOGY

The present disclosure relates to the technical field of medical instruments, and in particular to an ultrasonic probe rotation device.

### BACKGROUND

An ultrasound device, especially a fiber-type ultrasonic endoscope, has a slender structure so that an ultrasonic transducer fixed at an end of an ultrasonic probe can penetrate into a deeper part of the human body. Furthermore, the ultrasonic transducer is coupled to a driving and imaging system at the other end of the ultrasonic probe via a coaxial cable, and the ultrasonic transducer can freely rotate in the ultrasonic probe under the driving of an external motor so as to generate an annular image or a sectional image of a tissue section perpendicular to an axial direction. It can be seen that a device that drives the ultrasonic transducer to rotate freely and transmits a sensing signal generated by the ultrasonic transducer back will have a great effect on the imaging quality.

A rotating assembly commonly used in the ultrasonic probe of the prior art is usually made of different components mated along a radial direction. As shown in FIG. 1, which is a structural diagram of a first connector 1 and a second connector 2 of an ultrasonic probe rotation assembly of the prior art, during a mating process it is necessary to enable two rods of the first connector 1 and two rods of the second connector 2 to be arranged in a staggered manner, and during the rotating process the driving motion of the first connector 1 and the second connector 2 is achieved by the stagger of two of the rods. However, since the rotation assembly needs to be used under high-speed rotation, long-term use of it is prone to cause slipping off of some components in the rotation assembly and a phenomenon of rotational separation.

Therefore, it is necessary to design a novel ultrasonic probe rotation device to avoid the aforementioned problems existed in the prior art.

### SUMMARY

An objective of the present disclosure is to provide an ultrasonic probe rotation device to facilitate quick mate in order to installation and avoid rotational separation.

In order to achieve the aforementioned objective, the ultrasonic probe rotation device of the present disclosure includes:
a first connector including a first connector head and a shaft sleeve configured to surround the first connector head, wherein an end of the shaft sleeve is provided with several limiting grooves, and top ends of adjacent ones of the limiting grooves are coupled by a guide surface which is inclined to an axis of the shaft sleeve; and
a second connector for coupling to the first connector, the second connector including a second connector head and a rotating inner core configured to surround the second connector head, wherein the second connector head is adapted to mate with the first connector head, and the rotating inner core is disposed opposite to an end of the shaft sleeve;
wherein, during the rotation of the rotating inner core, an end of the rotating inner core is configured to abut against the guide surface and then moves toward the limiting groove by the guide surface and is locked into the limiting groove.

The beneficial effect of the ultrasonic probe rotation device of the present disclosure is that an end of a shaft sleeve of the first connector is provided with several limiting grooves, and top ends of adjacent ones of the limiting grooves are coupled by a guide surface. During the rotation of a rotating inner core of the second connector, an end of the rotating inner core is configured to abut against the guide surface and then moves toward the limiting groove by the guide surface and is locked into the limiting groove so as to facilitate quick installation and mating and to avoid occurrence of rotational separation.

Preferably, the end of the rotating inner core includes at least two protrusions configured to abut against the guide surface and then move toward the limiting groove and are locked into the limiting groove.

Further preferably, the number of the limiting grooves is no less than the number of the protrusions.

Preferably, the guide surface between the adjacent ones of the limiting grooves is configured to extend in a direction away from the axis of the shaft sleeve.

Further preferably, two of the guide surfaces coupled to a common limiting groove are arranged in mirror images of each other.

Preferably, each of the limiting grooves includes a first side wall and a second side wall that are disposed opposite to each other, a height of the first side wall is greater than that of the second side wall, an end of the guide surface is configured to intersect with the first side wall of one of the adjacent ones of the limiting grooves, and the other end of the guide surface is configured to intersect with the second side of another one of the adjacent ones of the limiting grooves.

Preferably, the second connector further includes a socket sleeve configured to surround the rotating inner core, and an inner diameter of the socket sleeve is configured to match with the outer diameter of the shaft sleeve.

Preferably, the ultrasonic probe rotation device further includes a locking part and a trigger part, wherein the locking part is disposed on an outer wall of the first connector, the trigger part is disposed on an outer wall of the second connector, and during a mating process of the first connector and the second connector, the trigger part acts on the locking part and is locked into the locking part.

Further preferably, the locking part includes an L-shaped channel and a movable lock structure, an end of the L-shaped channel is disposed on an end of the outer wall of the first connector to form an opening end, the other end of the L-shaped channel is disposed in correspondence with the movable lock structure, and a width of the L-shaped channel is set to allow the trigger part to pass through and conduct relative movement.

Further preferably, an inner diameter of the first connector is equal to an outer diameter of the second connector, so that after the mating of the first connector and the second connector is realized, at least a part of the outer wall of the first connector is configured to abut against a part of the outer wall of the second connector.

Further preferably, an elastic ring is sleeved onto the outer wall of the second connector to achieve a sealing effect between the first connector and the second connector.

Preferably, the ultrasonic probe rotation device further includes a rotation driving part that is in electrical contact with the rotating inner core.

### BRIEF DESCRIPTION OF THE DRAWINGS

FIG. 1 is a schematic structural diagram of a first connector and a second connector of an ultrasonic probe rotation assembly of the prior art;

FIG. 2 is a schematic structural diagram of a first connector according to an embodiment of the present disclosure;

FIG. 3 is a schematic structural diagram of a second connector according to an embodiment of the present disclosure;

FIG. 4 is a schematic structural diagram of another second connector according to an embodiment of the present disclosure;

FIG. 5 is a schematic structural diagram of another first connector of the present disclosure; and

FIG. 6 is a schematic structural diagram of an ultrasonic probe rotation device according to an embodiment of the present disclosure.

### DESCRIPTION OF THE EMBODIMENTS

In order to make the objectives, technical solutions and advantages of the embodiments of the present disclosure clearer, the following will clearly and completely describe the technical solutions in the embodiments of the present disclosure, and the described embodiments are a part rather than all of the embodiments of the present disclosure. All other embodiments obtained by those of ordinary skills in the art based on the embodiments of the present disclosure without creative efforts are within the claimed scope of the present disclosure. The technical or scientific terms used herein shall have the usual meanings understood by those of ordinary skill in the art to which the present disclosure belongs, unless otherwise defined. The “comprise(s)/comprising” or “include(s)/including” and the like similar words as used herein mean that the elements or articles appearing before the word encompass the elements or articles listed after the word and their equals, and do not exclude other elements or articles.

An embodiment of the present disclosure provides an ultrasonic probe rotation device to facilitate quick installation and mating and to avoid rotational separation.

The ultrasonic probe rotation device according to the embodiment of the present disclosure includes a first connector and a second connector.

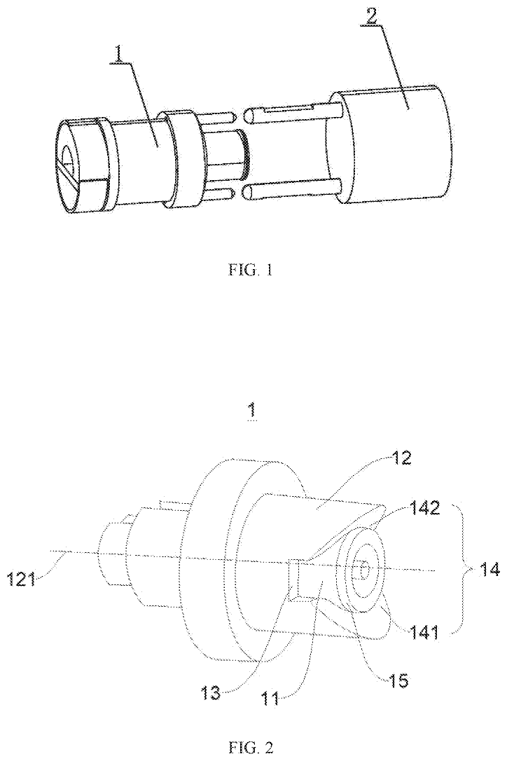

FIG. 2 is a schematic structural diagram of a first connector according to an embodiment of the present disclosure.

Referring to FIG. 2, the first connector 1 includes a first connector head 11 and a shaft sleeve 12 configured to surround the first connector head 11, an end of the shaft sleeve 12 is provided with several limiting grooves 13, top ends of adjacent ones of the limiting grooves 13 are coupled by a guide surface 14 which is inclined to an axis 121 of the shaft sleeve 12.

In some embodiments, referring to FIG. 2, the guide surface 14 between the adjacent ones of the limiting grooves 13 extends in a direction away from the axis 121 of the shaft sleeve 12.

In some embodiments, referring to FIG. 2, two of the guide surfaces 14 to the same limiting groove 13, i.e., a first guide surface 141 and a second guide surface 142, are mirror images of each other.

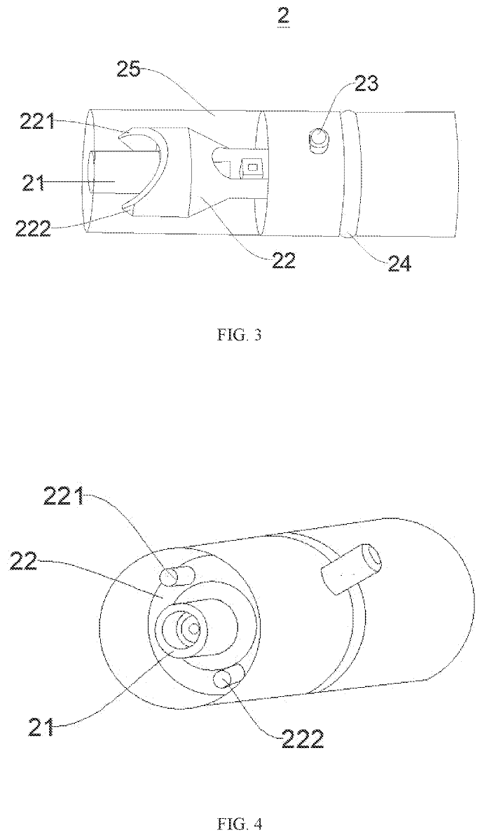

FIG. 3 is a schematic structural diagram of a second connector according to an embodiment of the present disclosure. FIG. 4 is a schematic structural diagram of another second connector according to an embodiment of the present disclosure.

Referring to FIGS. 2 to 4, the second connector 2 includes a second connector head 21 and a rotating inner core 22 configured to surround the second connector head 21, the second connector head 21 is adapted to the first connector head 11, and the rotating inner core 22 is disposed correspondingly to an end of the shaft sleeve 12.

In some embodiments, an outer diameter of the second connector head 21 is equal to an inner diameter of the first connector head 11 to achieve adaptation.

In some embodiments, during the rotation of the rotating inner core 22 and the mating process of the second connector head 21 and the first connector head 11, due to the guide surface 14 is inclined to the axis 121 of the shaft sleeve 12, an end of the rotating inner core 22 is configured to abut against the guide surface 14 and then moves toward the limiting groove 13 by the guide surface and is locked into the limiting groove 13 to facilitate quick installation and mating and to avoid rotational separation.

In some embodiments, referring to FIGS. 2 to 4, an end of the rotating inner core 22 includes a first protrusion 221 and a second protrusion 222. During the rotation of the rotating inner core 22 and the mating of the second connector head 21 and the first connector head 11, the first protrusion 221 and the second protrusion 222 contact the first guide surface 141 and the second guide surface 142, and then respectively slide toward the adjacent ones of the limiting grooves 13 and are respectively limited by the limiting grooves 13.

In some embodiments, the number of the protrusions at the end of the rotating inner core 22 is set to be at least 2.

In some embodiments, the number of the limiting grooves 13 is no less than the number of the protrusions.

In some embodiments, referring to FIGS. 2 and 3, the second connector 2 further includes a socket sleeve 25 configured to surround the rotating inner core 22, and an inner diameter of the socket sleeve 25 is adapted to match with the outer diameter of the shaft sleeve 12, so that the inner wall of the socket sleeve 25 can be attached to the outer wall of the shaft sleeve 12 to enhance the sealing performance of the ultrasonic probe rotation device and reduce interference from an external signal.

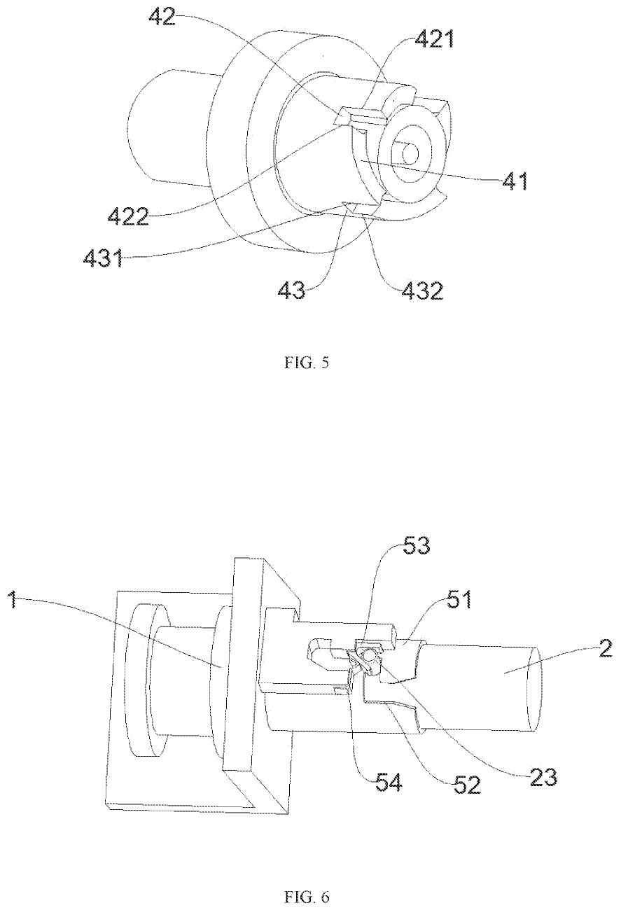

FIG. 5 is a schematic structural diagram of another first connector of the present disclosure.

As shown in FIG. 5, in an embodiment of the present disclosure, the ultrasonic probe rotation device includes a first connector and a second connector. The first connector includes a first connector head and a shaft sleeve configured to surround the first connector head, an end of the shaft sleeve is provided with several limiting grooves, and top ends of adjacent ones of the limiting grooves are coupled by a guide surface. The second connector is configured to mate with the first connector. The second connector includes a second connector head and a rotating inner core configured to surround the second connector head. The second connector head is adapted to mate with the first connector head, and the rotating inner core is disposed opposite to an end of the shaft sleeve. During the rotation of the rotating inner core, the end of the rotating inner core is configured to firstly abut against the guide surface so as to form a plurality of unlocked positions, and then moves toward the limiting grooves and is finally locked into the limiting grooves to form locked positions, so that the mating of the first connector and the second connector is achieved.

Referring to FIGS. 2 and 5, among the four limiting grooves as shown in FIG. 5, the top ends of the adjacent ones of the first limiting grooves 42 and the top ends of the second limiting grooves 43 are coupled by the first guide surface 41. Specifically, for the first limiting groove 42, the height of a first side wall 421 of the first limiting groove is greater than that of a second side wall 422 of the first limiting groove disposed opposite to the first side wall 421 of the first limiting groove, and for the second limiting groove 43, the height of a first side wall 431 of the second limiting groove is greater than that of a second side wall 432 of the second limiting groove disposed opposite to the first side wall 431 of the second limiting groove. The heights of the four side walls refer to the heights of the four side walls extending in a direction along the axis 121 of the shaft sleeve 12. An end of the first guide surface 41 is configured to intersect with the second side wall 422 of the first limiting groove, and the other end of the first guide surface is configured to intersect with the first side wall 431 of the second limiting groove.

FIG. 6 is a schematic structural diagram of an ultrasonic probe rotation device according to an embodiment of the present disclosure.

Referring to FIG. 6, the ultrasonic probe rotation device as shown in FIG. 6 further includes a trigger part 23, and a locking part that is composed of a V-shaped lock structure 53, an elastic member 54 and an L-shaped channel 52, wherein the locking part is disposed on the outer wall of the first connector 1. The V-shaped lock structure 53 and the elastic member 54 are combined to form a movable lock. The trigger part 23 is disposed on the outer wall of the second connector 2. During the mating process of the first connector 1 and the second connector 2, the trigger part 23 acts on the locking part (not labeled in the figure) and is clamped in the locking part (not labeled in the figure).

In some embodiments, referring to FIG. 6, an end of the L-shaped channel 52 is disposed at an end of the outer wall of the first connector 1 so as to form an opening end, the other end of the L-shaped channel is disposed in correspondence with the V-shaped lock structure 53 in the movable lock structure (not labeled in the figure), and the width of the L-shaped channel 52 allows the trigger part 23 to pass through and make relative movement therebetween. The V-shaped lock structure 53 is movably disposed on the outer wall of the first connector 1 and movably abuts against the elastic member 54. The trigger part 23 moves toward the V-shaped lock structure 53 through the L-shaped channel 52 and is limited by the V-shaped lock structure 53 and the elastic member 54 within an internal space surrounded by the V-shaped lock structure 53, thereby realizing the locking of the first connector 1 and the second connector 2.

In some embodiments, the inner diameter of the first connector 1 is equal to the outer diameter of the second connector 1, so that after the mating of the first connector 1 and the second connector 2 is realized, a part of the outer wall of the first connector 1 is attached to a part of the outer wall of the second connector 2.

In some embodiments, referring to FIG. 3, an elastic ring 24 is sleeved onto the outer wall of the second connector 2 so as to achieve a sealing effect between the first connector 1 and the second connector 2.

In some embodiments, the ultrasonic probe rotation device further includes a rotation driving part that is in electrical contact with the rotating inner core 22 to drive the rotating inner core 22 to rotate at a high speed.

Although the embodiments of the present disclosure have been described in detail above, it will be obvious to those skilled in the art that various modifications and changes can be made to these embodiments. However, it should be understood that such modifications and changes are within the scope and spirit of the present disclosure as described in the claims. Furthermore, the present disclosure described herein can have other embodiments and can be implemented or practiced in various ways.

What is claimed is:

1. An ultrasonic probe rotation device, comprising:
a first connector comprising a first connector head and a shaft sleeve configured to surround the first connector head, wherein an end of the shaft sleeve is provided with several limiting grooves, and top ends of adjacent ones of the limiting grooves are coupled by a guide surface which is inclined to an axis of the shaft sleeve; and
a second connector for coupling to the first connector, wherein the second connector comprises a second connector head and a rotating inner core configured to surround the second connector head, the second connector head is adapted to mate with the first connector head, and the rotating inner core is disposed opposite to an end of the shaft sleeve;
wherein, during the rotation of the rotating inner core, an end of the rotating inner core is configured to abut against the guide surface, and then moves toward the limiting groove by the guide surface and is locked into the limiting groove.

2. The ultrasonic probe rotation device according to claim 1, wherein the end of the rotating inner core comprises at least two protrusions configured to abut against the guide surface and then move toward the limiting groove and are locked into the limiting groove.

3. The ultrasonic probe rotation device according to claim 2, wherein the number of the limiting grooves is no less than the number of the protrusions.

4. The ultrasonic probe rotation device according to claim 1, wherein the guide surface between the adjacent ones of the limiting grooves is configured to extend in a direction away from the axis of the shaft sleeve.

5. The ultrasonic probe rotation device according to claim 4, wherein two guide surfaces coupled to a common limiting groove are arranged in mirror images of each other.

6. The ultrasonic probe rotation device according to claim 1, wherein each of the limiting grooves comprises a first side wall and a second side wall disposed opposite to the first side wall, a height of the first side wall is greater than that of the second side wall, an end of the guide surface is configured to intersect with the first side wall of one of the adjacent ones of the limiting grooves, and the other end of the guide surface is configured to intersect with the second side of another one of the adjacent ones of the limiting grooves.

7. The ultrasonic probe rotation device according to claim 1, wherein the second connector further comprises a socket sleeve configured to surround the rotating inner core, and an inner diameter of the socket sleeve is configured to match with the outer diameter of the shaft sleeve.

8. The ultrasonic probe rotation device according to claim 1, further comprising a locking part and a trigger part, wherein the locking part is disposed on an outer wall of the first connector, the trigger part is disposed on an outer wall of the second connector, and during a mating process of the first connector and the second connector, the trigger part acts on the locking part and is locked into the locking part.

9. The ultrasonic probe rotation device according to claim 8, wherein the locking part comprises an L-shaped channel and a movable lock structure, an end of the L-shaped channel is disposed on an end of the outer wall of the first connector to form an opening end, the other end of the L-shaped channel is disposed in correspondence with the movable lock structure, and a width of the L-shaped channel is set to allow the trigger part to pass through and make relative movement therebetween.

10. The ultrasonic probe rotation device according to claim 8, wherein an inner diameter of the first connector is equal to an outer diameter of the second connector, so that after the mating of the first connector and the second connector is realized, at least a part of the outer wall of the first connector is configured to abut against a part of the outer wall of the second connector.

11. The ultrasonic probe rotation device according to claim 8, wherein an elastic ring is sleeved onto the outer wall of the second connector to achieve a sealing effect between the first connector and the second connector.

12. The ultrasonic probe rotation device according to claim 1, further comprising a rotation driving part that is in electrical contact with the rotating inner core.
